(19) United States
(12) Patent Application Publication
(10) Pub. No.: US 2011/0182987 A1
(43) Pub. Date: Jul. 28, 2011

(54) PHARMACEUTICAL DOSAGE FORM FOR THE SITE-SPECIFIC DELIVERY OF MORE THAN ONE ACTIVE PHARMACEUTICAL INGREDIENT

(76) Inventors: Priya Bawa, Vereeniging (ZA); Viness Pillay, Sandton (ZA); Yahya Choonara, Lenasia (ZA)

(21) Appl. No.: 12/999,824

(22) PCT Filed: Jun. 3, 2009
(86) PCT No.: PCT/IB09/05830

§ 371 Date: Apr. 12, 2011

(30) Foreign Application Priority Data
Jun. 19, 2008 (ZA) 2007/11000

(51) Int. Cl. A61K 47/30; A61K 9/00; A61K 31/715; A61K 9/20; A61K 9/28; A61K 9/36; A61K 31/732; A61K 31/734; A61K 31/723; A61K 31/722; A61K 31/717; A61K 31/721; A61P 1/00; B82Y 5/00

(52) U.S. Cl. 424/464; 424/400; 514/54; 424/78.01; 424/474; 424/480; 514/55; 514/57; 514/59; 977/788

(57) ABSTRACT

This invention relates to a pharmaceutical dosage form for the site specific delivery of more than one active pharmaceutical ingredient to different sites in the human or animal body in the gastrointestinal tract. The dosage form has an outer polymeric layer incorporating a first active pharmaceutical ingredient which reacts to stimuli specific in the stomach, degrades, and releases the first active pharmaceutical ingredient in the stomach for absorption. The dosage form also has at least one inner polymeric layer incorporating a second active pharmaceutical ingredient which, once the outer layer has degraded, passes into the intestine where the polymers of the second layer degrade to release the second active pharmaceutical ingredient. The dosage form may have additional layers each incorporating active pharmaceutical ingredients for release in different portions of the intestine depending on the nature of the polymers.

### FIELD OF THE INVENTION

This invention relates to a pharmaceutical dosage form and, more particularly, to a pharmaceutical dosage form for the site-specific delivery of more than one pharmaceutical composition in a human or animal body.

### BACKGROUND TO THE INVENTION

The treatment of a number of medical conditions, particularly there those involving the gastrointestinal tract often require the administration of multiple active pharmaceutical ingredients (“APIs”) or drugs for local or systemic delivery, often to different portions of the gastrointestinal tract and more often than not in elevated doses.

By way of example, if treatment of the condition known as Irritable Bowel Syndrome and, more particularly, Ulcerative Colitis, is considered, a two-API treatment regime is recommended. The first API is intended for gastric delivery, preferably in the stomach of a patient and the second API is, preferably, released into and absorbed by the colon, or, alternatively, acts locally within the colonic region of the GIT. This means that the pharmaceutical dosage form, if taken orally, must be retained in the stomach for a period sufficient for the first API to be released into the stomach where it is absorbed. The remaining dosage form which then contains the second API must pass through the pyloric sphincter into and though the small intestine into the large intestine or colon without releasing an appreciable quantity of the second API. The said remaining dosage form, may also be formulated in manner that the second API is readily absorbed on entry to the proximal small intestine.

Previously such a condition was treated by administering orally, two separate tablets or capsules each containing a different API. The tablet containing the second API was coated so that it maintained its integrity in the stomach and small intestine but dissolved in the colon to release the second API. A number of patents and patent applications claiming protection for pharmaceutical dosage forms containing a single API and a means for either accelerating or delaying the release of the API have been filed. The most relevant of these known to the applicants are the following:
1) PCT patent application no. PCT/ US98/20779 which discloses a gastrointestinal drug delivery system for releasing a single drug or API in the gastrointestinal tract in a location and time dependent manner;
2) PCT patent application no. PCT/JP01/03229 discloses time release coached solid formulations for delivering a single drug or API in the gastrointestinal tract;
3) European patent application no. EP 1 275 381 which discloses a time release coated solid composition for oral administration of a drug to the lower digestive tract. This disclosure relates to the delivery of a single API;
4) PCT patent application no. PCT/GB2005/002977 discloses a composition for the oramucosal delivery of a single API within five minutes when applied to an oramucosal surface; and
5) United States patent application publication number US 2008/0193535 which, although published after the priority date of the current application provides an indication of the current state of the art with regard to the subject matter of the present invention which is the rapid delivery of a single API in the form of an allergen.

All of the above disclosures relate to the delivery of a single API and while such compositions are used in methods of treating a medical condition requiring the delivery of more than one API to different parts of the body they rely on accurate filling of prescriptions and also on the diligent cooperation of a patient for it is essential that a specified number of different tablets or capsules are taken either simultaneously or at prescribed intervals. When treating less sophisticated patients, insufficient care is often exhibited by the patient, resulting in one tablet being missed, often for a number of dosage times, and this renders the treatment ineffective.

### OBJECT OF THE INVENTION

It is an object of this invention to provide a pharmaceutical dosage form and, more particularly, to provide a pharmaceutical dosage form for the site-specific delivery of more than one pharmaceutical composition.

### SUMMARY OF THE INVENTION

In accordance with this invention there is provided a pharmaceutical dosage form for the site-specific delivery of more than one API, the dosage form comprising at least one outer layer containing at least one API for delivery to a first site in a human or animal body and at least one inner layer containing at least one API for delivery to a second site in the human or animal body, each layer having characteristics which, when subjected to specific stimuli unique to its delivery site, enable the release in said site of said API.

There is further provided for the dosage form to have at least one intermediate layer located between the outer layer and the inner layer, the intermediate layer containing at least one API for delivery to a site between the first and second sites.

There is also provided for the outer layer to be in the form of a shell which, in use, inhibits release of APIs contained in the inner layers until substantially all of the API in the outer layer has been released.

There is also provided for each of the layers to be platforms, preferably polymeric platforms and for each API to be incorporated into said polymeric platform.

There is also provided for the polymeric platforms to be manufactured from natural and/or synthetic polymers and for each API to be incorporated into said polymeric platform.

There is also provided for the natural polymers of the polymeric platform to be selected from polysaccharide polymers and, preferably, for the polysaccharide polymers to be selected from the group consisting of: chitosan, pectin, xanthan gum, sodium alginate, celluloses such as sodium carboxymethycellulose (CMC), hydroxypropylcellulose (HPC), hydroxyethylcellulose (HEC), hydroxypropylmethylcellulose (HPMC), and dextrans.

There is also provided for the synthetic polymers of the polymeric platform to include a standard hydrophilic polymer; alternatively a hydrophilic, swellable or erodible polymer; further alternatively a standard hydrophobic polymer; still further alternatively a hydrophobic swellable or erodible polymer; and still further alternatively a stimulus-responsive polymer; for the various polymers to include at least one of polyethylene oxide (PEO), polyvinyl alcohol (PVA), ethylcellulose (EC), poly(lactic) co-glycolic acids (PLGA), polylactic acids (PLA), polymethacrylates, polycaprolactones, polyesters and polyamides, and for the said polymers to be mixed, in use, with a co-polymer, alternatively for the polymers to be used on their own.

There is also provided for the or each API to be in the form of micro- and/or nanostructures and for these to be incorporated into a polymeric platform by mixing them with the polymer and/or other rate-modulating critical formulation adjuvants.

There is further provided for the site specific regions to which the APIs are delivered to be regions of the gastrointestinal tract, preferably the stomach and the colon and for the pharmaceutical dosage form to have gastrofloatable properties where it is, initially buoyant or becomes buoyant on the surface of gastric contents, alternatively gastrosinking properties where it is more dense than the gastric fluid in which case it sinks to the antrum of the stomach, further alternatively gastroswellable properties where the dosage form swells and prevents the rapid gastric emptying through the pyloric sphincter of the stomach based on swellable dimensions of the dosage form, and still further alternatively where it adheres, in use, to the wall of the stomach or another region of the GIT thus preventing premature gastric emptying, duodenal emptying, intestinal emptying, or colonic emptying depending on the site of adhesion.

There is also provided for the outer platform of the dosage form to dissolve in response to site-specific stimuli, preferably pepsin, in the stomach and, once dissolved, for the remainder of the said dosage form to move, in use, into and to pass through the small intestine to, eventually, enter the colonic region of the gastrointestinal tract where the inner platform of the dosage form dissolves in response to site-specific stimuli in the colonic region and release the API.

There is also provided for the inner platform of the dosage form to contain and release a compound/s that enhances absorption of the API in the colonic region.

There is also provided for the dosage form to release the API incorporated into each platform as the platform dissolves thus making the API available for absorption in the site in which it is released and/or making the API available to act locally at its target site.

There is further provided for the API to be selected from one or more of several APIs which are selected from the group consisiting of: anti-inflammatories, corticosteroids, antidiarrhoeals, opioids, immunosuppressives, antibiotics, antiemetics, antifungals, antivirals, antimalarials, anti-TB, antiretrovirals, antihypertensives, proteins, peptides, chemotherapeutics, diagnostic agents, probiotics, prebiotics, multivitamins, minerals, trace elements, and phytonutrients.

There is further provided for the polymers forming the polymeric platforms to be in situ crosslinked with an electrolyte or salt which is incorporated into the pharmaceutical dosage form, the electrolyte or salt being selected from the Hofmeister Series of salts and operable to retard the release of APIs from the pharmaceutical dosage from any or all of the platforms and or glutaraldehyde and formaldehyde.

There is also provided for the polymer to be crosslinked by using microwave radiation, UV radiation or chemical crosslinking.

There is also provided for the operatively innermost layer of the pharmaceutical dosage form to be at least one in situ crosslinked polymer forming a single discrete pellet containing at least one API embedded therein, or for the operatively innermost layer of the pharmaceutical dosage form to have a number of in situ crosslinked polymers and for the polymer or polymers to form a polymer matrix of various stimuli-responsive polymers and/or other critical formulation adjuvants and desired permutations depending on the nature of the polymer or polymers selected.

There is also provided for the dosage form to be formed by mixing a polymer in various concentrations, a pharmaceutical excipient, preferably a lubricant such as magnesium stearate, and/or a binder such as carboxymethylcellulose (CMC) and/or a crosslinking agent such as a desired salt, and at least one active ingredient in at least one of the components of the dosage form.

There is also provided for the release of the or each API from the outer polymeric layer of the pharmaceutical dosage form to be governed by the crosslinking agent employed, the degree of ionization of the crosslinking agent, the solution pH, the ratio of dry polymer to pepsin, and the degree of crosslinking.

There is also provided for the innermost polymeric platform to be configurable to suit a number of applications and administration methods, for the innermost polymeric platform to be embedded within the outermost polymeric platform, preferably a low-density, gastrofloatable platform so that, in use, APIs from either polymeric platform can be released over a desired period of time, preferably in a phase-controlled site-specific manner which may be rapid, alternatively slow, as a result of variations in the diffusion pathlengths created within the polymeric platforms.

There is also provided for a pharmaceutically active compound to be formulated into at least one disc and for the disc to be surrounded by a number of the same or alternating polymeric layers.

There is also provided for the outer polymeric platform to be in the form of a shell which, wholly or partly encapsulates an inner tablet-like component, the outer polymeric platform thus allowing the release of a first API in one region of the gastrointestinal tract, in particular the stomach, in response to specific stimuli in said region of the gastrointestinal tract, in particular pepsin.

There is also provided for the composition of the shell to comprise various natural and synthetic polymers, for said polymers to be selected from the group consisting of chitosan, gelatin and polyacrylamide as well as crosslinking agents from among the group comprising sucrose-6-1′-diacrylate.

There is further provided for the outer shell to be adhered to the inner tablet-like component using polymers with adhesive properties such as but not limited to polymers or compounds from among the group comprising polyvinylalcohol (PVA).

There is also provided for the tablet-like component to be comprised of crosslinked API-loaded granules dispersed within a matrix of various natural and synthetic polymers e.g. pectin, polyethylene oxide (PEO), and xanthan gum, for the granules to comprise natural polysaccharide polymers, preferably selected from the group consisting of alginate, pectin, xanthan gum or chitosan that are responsive to specific enzymes in various regions of the gastrointestinal tract, in particular the colon. Examples of such polymers include polysaccharide polymers that are susceptible to digestion/cleavage by colonic enzymes such as β-glucosidases, pectinases and other polysaccharidases, and for the granules to be crosslinked with various electrolytes/salts or in particular multivalent salts such as the tripolyphosphates.

There is further provided for the tablet-like matrix to be in situ crosslinked using various crosslinking agents such as electrolytes/salts.

There is also provided for the tablet-like component to be coated with a pH responsive, alternatively pH-independent, coating, solution or at least one hydrophobic polymer latex selected from the group consisting of: ethylcellulose, or cellulose acetate phthalate. Such coating solutions may be aqueous dispersions or may be dispersed in solvents such as acetone or ethanol, and for hydrophobic polymers to be dispersed within the matrix of the tablet-like component.

There is also provided for the pH responsive or pH-independent coating, solution or hydrophobic polymer latex to be applied to the pharmaceutical dosage form alone or in combination.

There is also provided for the coatings to be combined with various polysaccharide or enzyme responsive polymers in various ratios and combinations to form a desired pH/time/enzyme responsive coating.

There is also provided for the combination of coating solutions and polymers to be selected so as to render a polymeric component of the pharmaceutical dosage form pH responsive in use, thus facilitating precise delivery of an API to a desired site of action or absorption. Alternatively there is provided for the combination of coating solutions and polymers to be selected so as to render a polymeric component of the pharmaceutical dosage form responsive to one or more enzymes present in a desired site of action or absorption thus facilitating precise delivery of an API to a desired site of action or absorption. Further alternatively there is provided for the combination of coating solutions and polymers to be selected to degrade within a specific region of the human or animal body in a time dependent manner thus facilitating precise delivery of an API to a desired site of action or absorption.

There is further provided for the inner polymeric layer of the pharmaceutical dosage form to be tablet-shaped and for the tablet to be formed by compressing granules prepared by wet or dry granulation of a polysaccharide polymer or a combination of polymers and the API.

There is also provided for the inner polymeric layer of the pharmaceutical dosage form to be tablet-shaped and for the tablet to be formed by compressing granules, prepared by wet granulation or dry granulation of a polysaccharide polymer or a combination of polymers with a single or combination of crosslinking agents such as multivalent salts or other chemical reagents, using various solvents such as de-ionized water or ethanol, and the API.

There is further provided for the granules to be dispersed, for the granules to be prepared by wet granulation, and for the granules to be coated with a pH responsive or pH-independent coating solution or various hydrophobic polymer latexes which may be applied alone or in combination or not in a matrix of a single polymer or a combination of polymers.

There is also provided for the inner polymeric layer of the pharmaceutical dosage form to be tablet-shaped and for the tablet to be formed by direct compression of polymeric components of the formulation.

There is further provided for the coating or coatings to be combined with various polysaccharide or enzyme responsive polymers in various ratios and combinations to form a unique pH/time/enzyme responsive coating.

There is further provided for altering the micro-environment of the outer polymeric shell to facilitate an optimum environment for the chitosanolytic activity of pepsin, in use, thus improving the enzymatic responsiveness of the outer polymeric shell, ensuring sufficient or complete and site-specific delivery of the API. Alternatively there is provided for altering the micro-environment of the outer polymeric shell by adding various alkaline solutions such as sodium hydroxide solutions of various concentrations, ammonium hydroxide solutions of various concentrations; or by employing salts directly such as sodium bicarbonate and/or sodium carbonate.

There is also provided for the charge densities of the relevant polymers and crosslinking salts of the said pharmaceutical dosage form to be governed by the solution pH, with a lower solution pH producing, in use, a sufficient decrease in the degree of ionization of the crosslinking salt which results in polymeric crosslinking weakening between the polymer/s and crosslinking agent/s to facilitate the swelling of the outer polymeric layer of the pharmaceutical dosage form and allow for diffusion of fluid along with pepsin into the layer and cause cleavage and/or degradation of chitosan which results in API release from the pharmaceutical dosage form.

### BRIEF DESCRIPTION OF THE FIGURES

Embodiments of the invention will be described below by way of example only and with reference to the accompanying figures in which:

FIG. 1 is a graphical analysis of profiles showing drug release from crosslinked and non-crosslinked pectin AM 901 in simulated gastric fluid over a period of 24 hours;

FIG. 2 is a graphical analysis of profiles showing drug release from crosslinked and non-crosslinked pectin AMID CF 005 in simulated gastric fluid over a period of 24 hours;

FIG. 3 is a graphical analysis of profiles showing drug release from crosslinked and non-crosslinked pectin AMID CF 020 in simulated gastric fluid over a period of 24 hours;

FIG. 4 is a graphical analysis of profiles showing drug release from crosslinked and non-crosslinked pectin AM 901 in simulated intestinal fluid over a period of 24 hours;

FIG. 5 is a graphical analysis of profiles showing drug release from formulations incorporating three different in situ crosslinking agents namely zinc sulphate, aluminium chloride or barium chloride;

FIG. 6 is a graphical analysis of profiles showing drug release of formulations incorporating various polymers namely hydroxypropylmethylcellulose, poly(ethylene oxide) or hydroxyethylcellulose;

FIG. 7 is a graphical analysis of profiles showing drug release of formulations consisting of granules in various ratios of alginate, chitosan and ZnSO₄ in simulated gastric fluid;

FIG. 8 is a graphical analysis of profiles comparing drug release from formulations including Eudragit® or de-ionised water as a granulation solvent;

FIG. 9 is a graph showing profiles for drug release of chitosan/citrate films in SGF and SIF;

FIG. 10 is a graph showing profiles for drug release of gelatin/chitosan films in SGF with and without pepsin;

FIG. 11 is a graph showing profiles for drug release of gelatin films in SGF with and without pepsin;

FIG. 12 is a graph showing profiles for drug release of cross-linked chitosan shells in SGF with and without pepsin;

FIG. 13 is a graph showing profiles for drug release of non cross-linked chitosan shells in SGF with and without pepsin;

FIG. 14 is a schematic diagram of the proposed mechanism of drug release from an in situ crosslinked stimuli-responsive pharmaceutical dosage form;

FIG. 15 is a flow diagram describing the order of events occurring for the release of APIs from a in situ crosslinked and stimuli-responsive pharmaceutical dosage form; and

FIG. 16 is a schematic diagram of configurative variations of an in situ crosslinked and stimuli-responsive pharmaceutical dosage form.

### DESCRIPTION OF THE PREFERRED EMBODIMENTS

The oral route is the most common and convenient method of drug administration and more than 60% of marketed drugs are used orally (Masaoka et al., 2006). Prolonged release drug delivery systems typically provide significant benefits over immediate release formulations, including greater effectiveness in the treatment of chronic conditions, reduced side-effects and greater patient compliance due to a more simplified dosing schedule (Verma et al., 2002). There has also been increased emphasis on ways to deliver or activate drugs at specific sites in the body in order to reduce side-effects and increase the drugs pharmacological response. Site-specific drug delivery is proposed to be achievable by using implantable pumps, adhesive patches impregnated with drugs, vesicle enclosed drugs, drug carriers and prodrugs.

Gastro-retentive dosage forms may be beneficial for the site-specific delivery of drugs in the upper gastrointestinal tract to treat local pathology in the stomach e.g. peptic and duodenal ulcers and/or to allow a less frequent drug administration. A prolonged period of retention in the stomach may be beneficial for drugs most effectively absorbed locally in the stomach. The gastric emptying time normally averages 2-3 hours through the major absorption zones of the stomach and upper part of the intestine. This relatively brief gastric emptying time may result in incomplete drug release and diminished efficacy of the administered dose (Kim and Singh, 2000).

Gastrofloatable drug delivery systems have emerged as an attractive approach to achieving prolonged drug release by increasing the gastric residence time of the drug. The concept involves a system that has a bulk density lower than the gastric fluid thus remaining buoyant in the stomach for a prolonged period of time (Kim and Singh, 2000; Streubel et al., 2006). During this time, gradual drug release occurs at a desired rate. Prolonging the residence time of the delivery system in the stomach offers numerous advantages, especially for drugs that have a narrow absorption window, drugs with a stability problem in the intestine, or for localized gastric action (Kim and Singh, 2000; Garg and Sharma, 2003).

Even though these buoyant systems possess the inherent ability for gastric retention, they rely more on the presence of food to retard their gastric emptying. Therefore, only when the delivery system is independent of meal size will it be suitable for patients with a wide range of eating habits (Singh and Kim, 2000). Buoyant drug delivery systems that allow for optimum drug release and absorption without food being a prerequisite could be immensely beneficial for patients that have no regular food intake. Drug bioavailability will also not be dependent on whether or not the patient has eaten. According to Arora et al. (2005) the resting volume of the stomach is between 25 and 50 mL. Therefore, any system that enters the stomach will come into contact with this aqueous medium and remain buoyant for a certain period of time independant of whether or not food is ingested.

A relatively new approach of achieving site-specific drug delivery is by employing so-called ‘smart’ polymers. These polymers are capable of responding to small changes in the pH, temperature, electric or magnetic fields of the environment and can undergo fast, reversible changes in their microstructure. The incorporation of these stimuli-responsive polymers into drug delivery systems would translate a chemical signal (e.g. presence of the substrate) into an environmental signal (e.g. pH change) and then into a mechanical signal viz. shrinking or swelling of the hydrogel and controlled drug release (Galaev and Mattiasson, 1999). An effective approach of controlling the drug release rate from a hydrogel is to change the cross-linking density of the matrix by using varying exposure times of the polymer to cross-linking agents, varying the concentration of the cross-linking agent or by employing degradable hydrogels (Patil et al., 1997; Ay et al., 2007).

Several enzymatically degradable cross-linking agents that are capable of completely digesting swollen hydrogels in the gastro-intestinal tract are known. For example, azo cross-linkers can be degraded by azoreductase, and albumin modified with glycidyl acrylate can be degraded by a variety of proteolytic enzymes. These cross-linking agents are however not suitable for site-specific drug delivery (Park, 1998). A biodegradable cross-linking agent, sucrose-6-1′-diacrylate (SDA), was used to cross-link poly(acrylamide) in varying ratios. The study demonstrated that pepsin and lipase, both with acidic pH optima and both present in the stomach, were effective at catalyzing SDA hydrolysis and could be considered as catalysts for hydrogel degradation (Patil et al., 1997). The human stomach and small intestine contain roughly 10³-10⁴ colony forming units (CFU/mL) and this number increases dramatically on entry to the colon. By incorporating these stimuli-responsive polymers into a drug delivery system, the drug is ensured to be released only in response to specific stimuli, in particular pepsin, thereby ensuring site-specific drug delivery in the stomach.

Colonic bacteria ferment a wide range of substrates e.g. polysaccharides, mucopolysaccharides etc (Friend, 2005). These bacterial enzymes can be used as the basis for a stimuli-responsive system, as they will allow the degradation of polymeric matrices and trigger drug release only in response to these enzymes. Selectively delivering drugs to the colon has several benefits: 1) allows the local treatment of a colonic disease e.g. ulcerative colitis, Crohn's disease or colon cancer, 2) allows dose reduction as the drug can directly act on the diseased site, 3) result in a reduction in undesirable and potentially harmful side-effects resulting from systemic absorption, 4) it is useful for the administration of drugs that are an irritant to the gastric mucosa e.g. NSAID's or for drugs that are degraded by gastric juices or gastric enzymes e.g. proteins and peptides, 5) drugs reside longer in the colon than at other digestive organs, therefore, the time for drug absorption becomes prolonged and the total bioavailability of the drug increases.

Materials and Methods

Materials

Pectin AM 901 (LM) (apple pectin) DE 38-44%; Pectin AMID CF 005 (LM) (amidated citrus pectin) DE 33-39%, DA 11-17%; Pectin AMID CF 020 (LM) (amidated citrus pectin) DE 25-31%, DA 19-23%; Zinc Sulphate, Magnesium Stearate, GENU® Pectin type LM 102 AS, Aluminium chloride hexahydrate, Diphenhydramine HCl (Aldrich), Barium Chloride 2-hydrate (Saarchem), Zinc Sulphate (Rochelle), Magnesium stearate, Sodium Alginate Protanal® (BioPolymer), Polyox® WSR-303, Hydroxypropylmethylcellulose (Sigma), Natrosol® (Hercules), Chitosan, Eudragit S100.

Preparation of in Situ Crosslinkable Tablets with Varying Grades of Pectin

In the initial study, three different grades of pectin were analyzed for their effective in situ crosslinking ability in the gastric and intestinal region. Zinc sulphate (crosslinking agent) was ground using a pestle and mortar and combined with magnesium stearate, diphenhydramine HCl (model drug) and pectin. This process was performed with various grades of pectin in a 2:1 ratio (pectin:salt). The powders were compressed into tablets by direct compression into tablets of 13 mm diameter and 5 mm width using a benchtop hydraulic press. Separate batches of tablets comprising no crosslinking agents were prepared to compare the effects on drug release.

Evaluation of the Influence of Various Salts Prepared by Direct Compression on Drug Release

Once the desirable grade of pectin was identified, different salts were evaluated for their crosslinking ability. Three sets of formulations were prepared comprising each crosslinking agent namely zinc sulphate, aluminium chloride and barium chloride. Each tablet comprised the following: pectin and salt in a 1:1 ratio, diphenhydramine HCl, and 1% magnesium stearate. Tablets were compressed at a force of 5N using a Beckman Hydraulic Press® (13 mm in diameter and 5 mm in width). Dissolution studies were performed in simulated gastric fluid (SGF) (pH 1.2; 37° C.) and were analyzed for drug content using UV spectroscopy.

Evaluation of the Influence of Eudragit® on Drug Release

Once the desirable salt for in situ crosslinking was identified i.e. BaCl₂, a further evaluation was conducted by including Eudragit® (a hydrophilic pH-dependant polymer) in the formulation and determine its influence on drug release. A control formulation set consisting of pectin and BaCl₂ in an 11:1 ratio, diphenhydramine HCl (model drug) and magnesium stearate was produced and compared to a test formulation set incorporating Eudragit® L100. These were prepared by direct compression at 5N and underwent dissolution studies in SGF (pH 1.2; 37° C.).

Evaluation of the Influence of Various Hydrophilic and Hydrophobic Polymers on Drug Release

Three different polymers namely hydroxypropylmethylcellulose (HPMC), Poly(ethylene oxide) (PEO), and hydroxyethylcellulose (HEC) were incorporated into three formulation sets each comprising of pectin and BaCl₂ in a 10:1 ratio, diphenhydramine HCl (model drug), Eudragit® L100 and magnesium stearate. The tablets were compressed at 5N and underwent dissolution studies in both SGF (pH 1.2; 37° C.)) and simulated intestinal fluid (SIF) (pH 6.8; 37° C.) and were analyzed for drug content using UV spectroscopy.

Evaluation of the Influence of the Proportion of Crosslinking Agent to Polymer on Drug Release

In order to determine the effect that the ratio of crosslinking agent (salt) to polymer (alginate) had on drug release four different formulation sets were produced. Each set comprised of formulations prepared as either direct compression of dry powders or wet granulation prior to compression. Granules were prepared by wet granulation (2 mm sieve) and consisted of diphenhydramine HCl (model drug), sodium alginate, chitosan and ZnSO₄ in ratios of 2:1, 3:1, 4:1 and 5:1. De-ionized water was used as a solvent and the granules were dried for 12 hours at 40° C. The direct compression blend consisted of pectin and BaCl₂ (2:1), PEO, Eudragit® L100 and magnesium stearate. The prepared granules were then combined with and dispersed within the direct compression blend and compressed at 8N. All samples underwent dissolution studies in SGF (pH 1.2; 37° C.) and were analyzed for drug content using UV spectroscopy.

Once establishing the desirable ratio of crosslinker:polymer (alginate: chitosan: ZnSO₄ in a 4:1:1 ratio was selected), different approaches to the wet granulation method were evaluated to ensure the highest retardation of drug release in the gastric environment. Two formulation sets were prepared consisting of diphenhydramine HCl (model drug), alginate: chitosan: ZnSO₄ (4:1:1) in the form of granules and pectin, BaCl₂, Eudragit® L100, PEO and magnesium stearate in the direct compression blend. In the first set of formulations an enteric coating latex solution of Eudragit S100® was used as a solvent for granulation and de-ionized water was used as a granulation solvent for the second set of formulations. The granules were allowed to cure for 15 minutes after which a Eudragit S100® solution was sprayed onto the granules which were then allowed to dry at 40° C. for 12 hours before compression at 8N using a Carver Press®.

Formulation of a Low-Density pH-Responsive Polymeric Component

The low-density polymeric component was formulated by the casting/solvent evaporation technique. Briefly, a 10% w/v chitosan solution was prepared in 4M acetic acid. 2 g of the model drug was added to this solution and allowed to stir for 30 minutes to ensure all drug was dissolved. The above solution was allowed to stand, not stirring for a further 30 minutes to ensure that all trapped air bubbles had been removed.

Polystyrene trays with wells 13 mm in diameter were lubricated. 1 mL samples of the solution were placed in each well. The samples were allowed to dry under a fume hood for 48 hours at room temperature until constant weight.

The dried chitosan films were then crosslinked by soaking each film in an aqueous solution of tri-sodium citrate. The crosslinking conditions were as follows: a 10% w/v aqueous solution of tri-sodium citrate, solution pH of 5 and a crosslinking time of 1 hour. The crosslinked chitosan/citrate films were then washed with distilled water and placed on a glass petri dish and allowed to dry under the fume hood for a further 24 hours, at room temperature.

Evaluation of Gelatin for Responsiveness to Pepsin

A 21% w/v solution of gelatin was prepared by dissolving gelatin in water. A 20% w/v chitosan solution was prepared by dissolving chitosan in 1M acetic acid. 76 mL of the gelatin was combined with 20 mL of the chitosan solution and mixed thoroughly. Model drug was dissolved in this solution. 1 ml aliquots were placed in pre-lubricated cylindrical moulds and were allowed to dry under a fume hood. The resulting formulations were tested in simulated gastric fluid without and without pepsin.

A similar formulation to the above was prepared however chitosan was excluded. Samples were allowed to dry under a fume hood and were tested in SGF with and without pepsin.

Alterations of the Micro-Environment to Enhance Chitosanolytic Activity of Pepsin

Research has shown that optimum chitosanolytic activity of pepsin occurs at a pH of 4.5. Since the pH of the gastric environment rarely reaches this pH, investigations into altering the micro-environment of the chitosan films was carried out. Also, inclusion of a plasticizer into chitosan solutions was investigated in order to produce a more consistent, easily removable and more robust chitosan shell. A 10% w/v solution of chitosan was produced in 1M acetic acid. To this model drug was included. Plasticizer viz. glycerol in a 2:1 ratio of chitosan weight to glycerol was included. Sodium bicarbonate was added to produce a pH of 5.5. A pH higher than 7 would result in precipitation of chitosan out of the solution. 1 mL aliquots were placed in prelubricated polystyrene trays and were allowed to dry in an oven at 40° C. for 24 hours. The shells were crosslinked in a 10% w/v zinc sulphate solution and again allowed to dry. Crosslinked shells were then washed to remove surface drug and salts. Drug release studies were conducted on the cross-linked and non cross-linked shells in SGF with and without pepsin.

In Vitro Drug Release Studies

In vitro dissolution studies were conducted in a rotating paddle apparatus in SGF (pH 1.2; 37° C.) and SIF (pH 6.8; 37° C.). Samples of 5 mL were withdrawn every hour for the first 12 hours and again at 24 hours and analyzed by UV spectrophotometry.

Results and Discussion

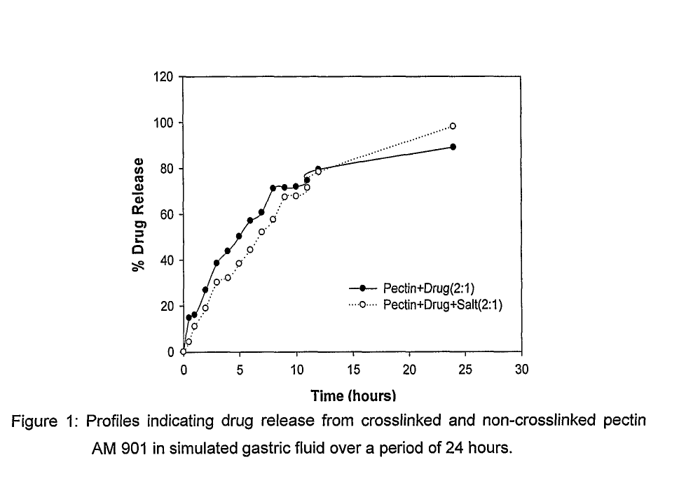

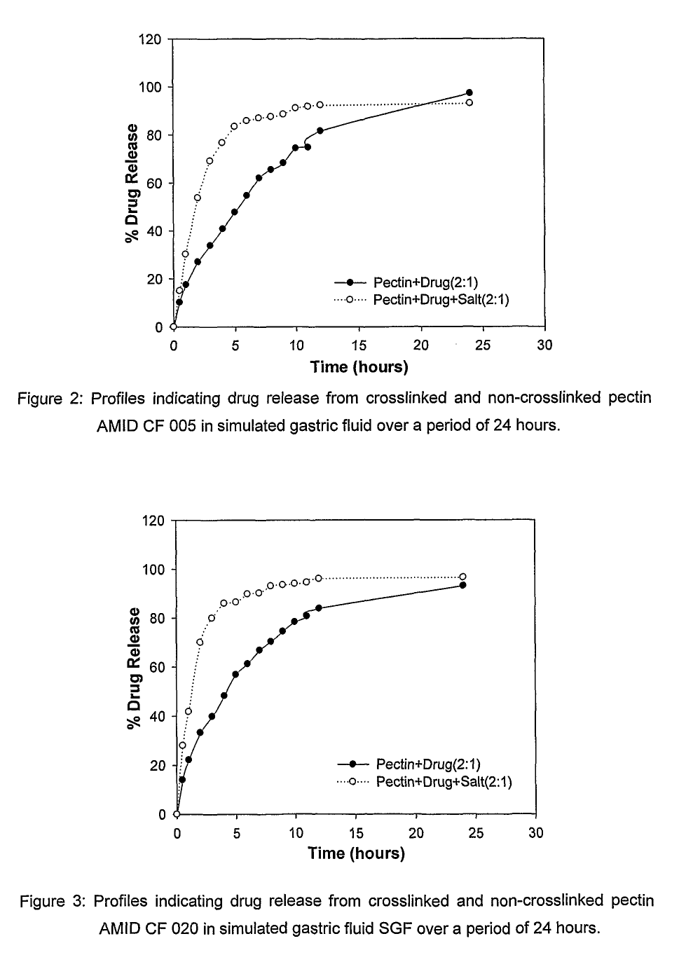

Drug release in SGF (pH 1.2; 37° C.) displayed a steady increase in absorbance with time for all grades of pectin (FIGS. 1, 2 and 3). Pectin AM 901 that was in situ crosslinked with ZnSO₄ showed a retardation of drug release when compared to the same grade of pectin without salt (43.7% vs. 56.16% after 6 hours), however Pectin AMID CF 005 and Pectin AMID CF 020 in situ cross-linked with ZnSO₄ showed a greater drug release compared to formulations without crosslinker.

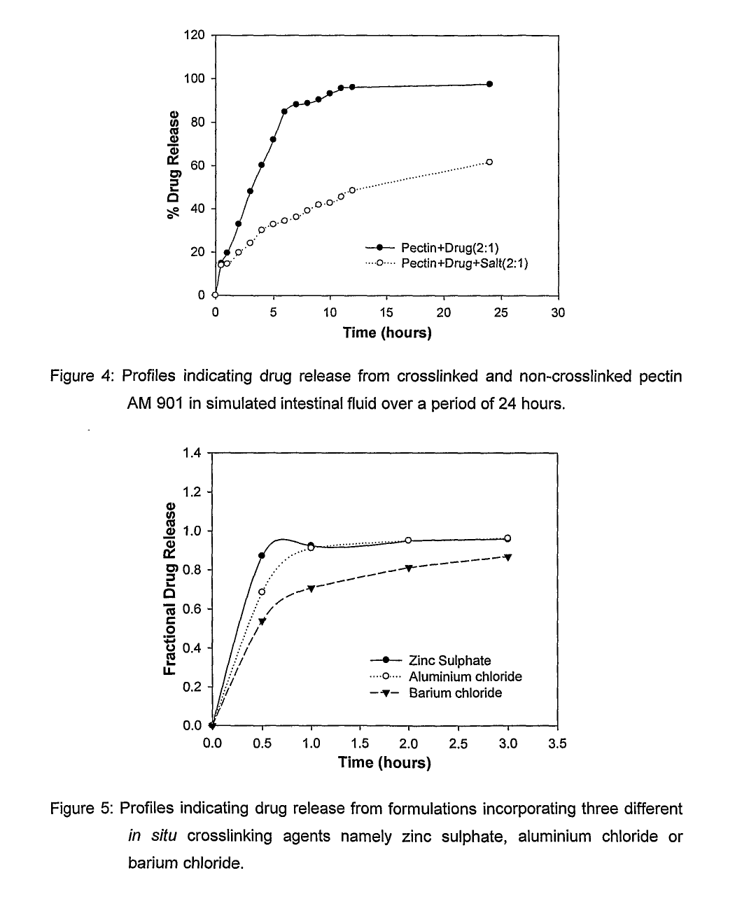

Drug release in SIF (pH 6.8; 37° C.) showed similar results to studies conducted in SGF (pH 1.2; 37° C.) (FIG. 4). Pectin AM 901 showed retardation in drug release when in situ crosslinked. Pectin AMID CF 005 again showed greater drug release when incorporated with ZnSO₄. Pectin AMID CF 020 crosslinked and non-crosslinked showed similar drug release for the first 5 hours however after 6 hours the crosslinked pectin had a lower drug release compared to non-crosslinked Pectin AMID CF 020.

Since the average gastric transit time is approximately 2 hours, the first 2 hours after drug administration remains the most important when analyzing drug release specifically in the gastric region. Therefore, the first 2 hours of in vitro drug release studies also remain the most important when attempting to limit drug delivery in the stomach. In this study formulations that incorporated ZnSO₄ and Al₂Cl₃ both showed a 95% drug release in SGF in 2 hours. BaCl₂ however showed a significantly lower drug release in this time (81%). Even though this value is not within the acceptable 0-10% drug release range, it still identifies BaCl₂ as the most efficient salt for in situ crosslinking (FIG. 5).

The control formulation set devoid of Eudragit® L100 showed a 37% drug release in the first 2 hours of dissolution studies. Complete drug release was only achieved after 24 hours of dissolution testing. Formulations comprising Eudragit® L100 had a 27% drug release in the first 2 hours and complete drug release was achieved after 24 hours.

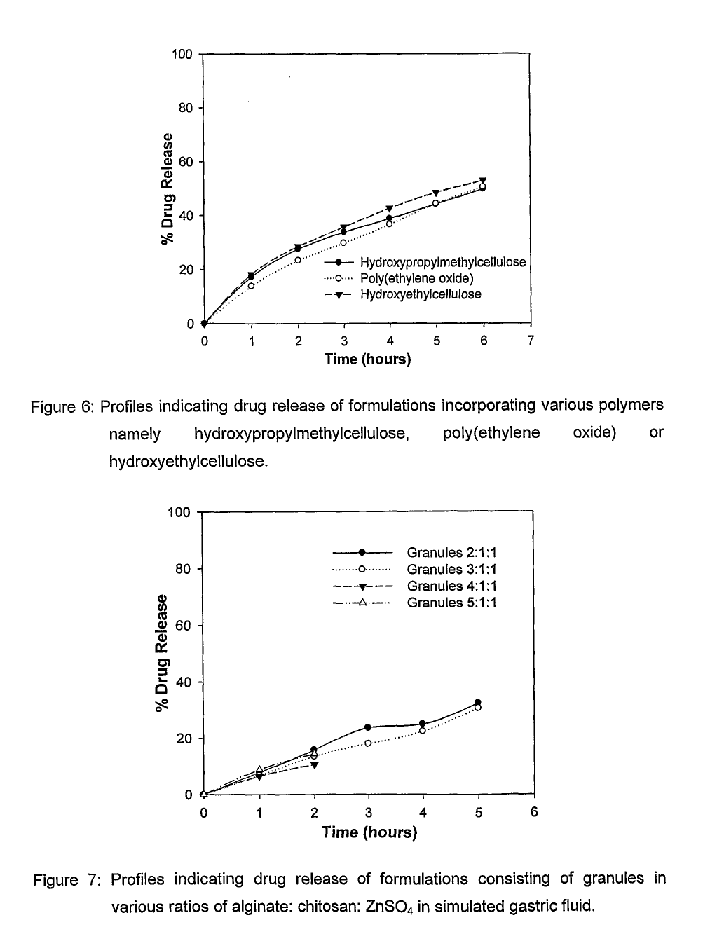

On determination of the effect that various polymers had on drug release in SGF it was found that HPMC and HEC both provided 28% drug release in 2 hours. After 6 hours HPMC provided 50% drug release and HEC provided 52% drug release in the same time period. PEO provided 23% drug release in 2 hours and 51% drug release in 6 hours (FIG. 6).

In SIF, PEO and HPMC both provided 21% drug release however HEC provided 29% drug release in this medium after 2 hours. From these results it can be concluded that PEO provided the most efficient retardation of drug release in both SGF and SIF when compared to HPMC and HEC.

When granules were produced of alginate: chitosan: ZnSO₄ in various ratios, dissolution studies conducted in SGF showed that the granules that were in a 2:1:1 ratio and 3:1:1 ratio had a similar release profile e.g. after 5 hours of dissolution granules in a 2:1:1 ratio provided 32.2% drug release and granules in a 3:1:1 ratio provided 30.4% drug release. The granules in the 4:1:1 ratio showed the best retardation of drug release with 10.6% drug released in the 2 hours and granules in a 5:1:1 ratio showed 14.4% drug release in the same time period (FIG. 7).

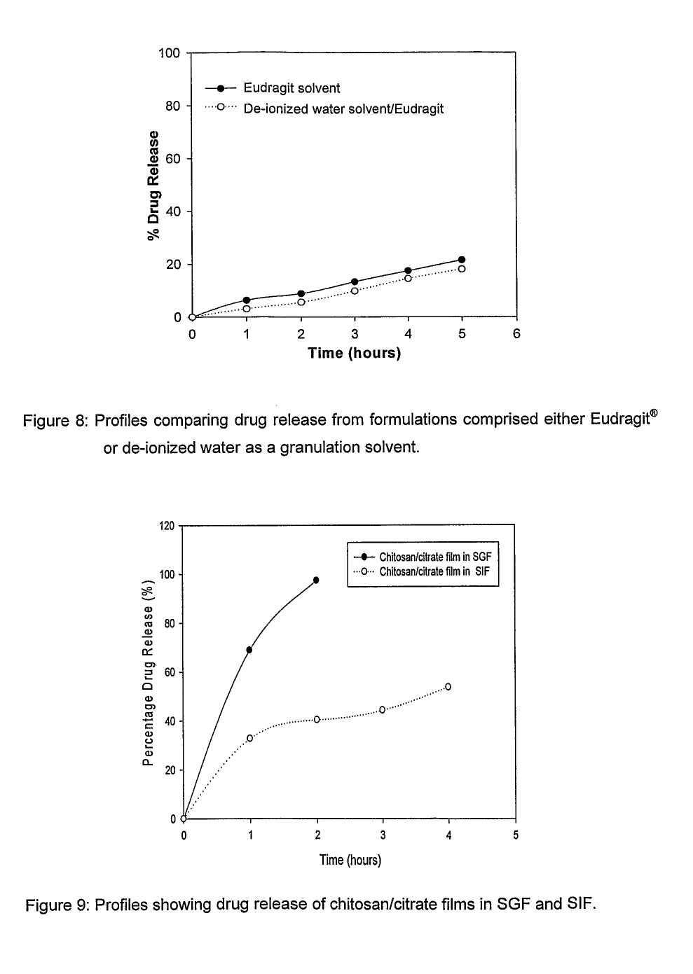

With granules in the desirable ratio of 4:1:1, different granulation solvents were used. Granules prepared using a Eudragit® S100 latex as a solvent had a 8.9% drug release in the first 2 hours and 21.5% drug release in 5 hours. When granules were prepared using de-ionized water as a solvent and consequently spraying on the latex solution, 5.6% drug release was achieved in the first 2 hours and 18% drug release in 5 hours (FIG. 8).

On observation of the chitosan/citrate films in the relevant dissolution media it was apparent that the films remained buoyant in SGF for the period of in vitro release studies, this however was not the case in SIF where the films immediately sank to the bottom of the dissolution vessel. After the first hour of dissolution studies in SGF the films had swollen significantly and after the second hour it had completely disintegrated. The films in SIF were still intact even after 5 hours of testing and showed no swelling.

From FIG. 9 it can be seen that crosslinked chitosan films are stimuli-responsive to conditions in the stomach. More specifically it is responsive to the pH of the stomach. In SIF the chitosan films had only 53% drug release compared to the 100% drug release it experienced in SGF at pH 1.2.

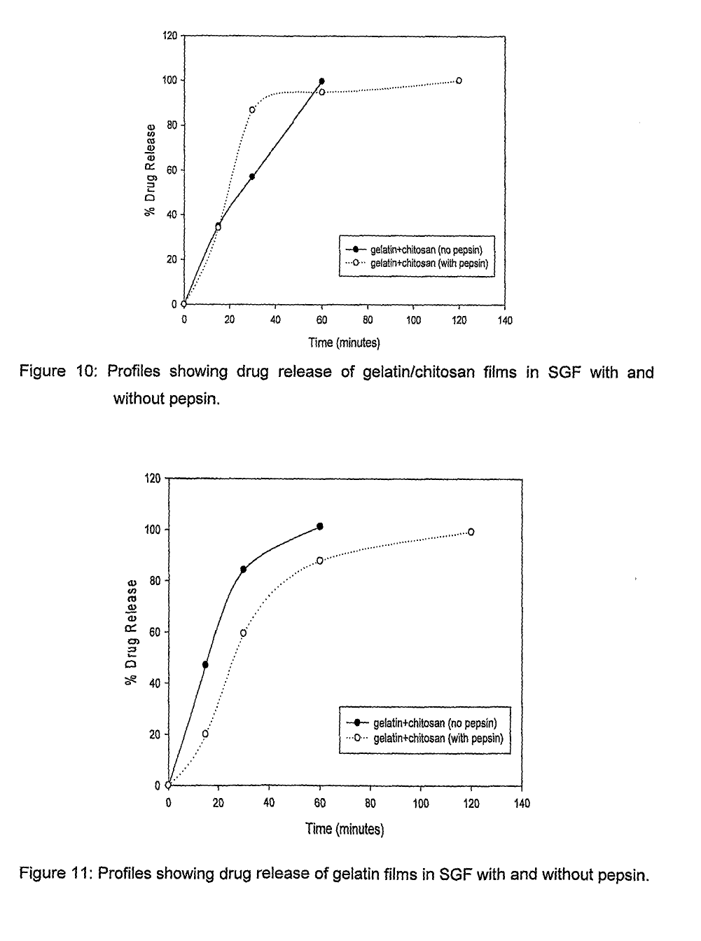

From FIG. 10 it can be seen that drug release from the formulation of a combination of chitosan and gelatin showed that in simulated gastric fluid containing pepsin drug release from the formulation was higher than in SGF without pepsin. However, in the first hour all drug was released.

Gelatin has shown not to be responsive to pepsin in SGF (FIG. 11). The responsiveness of the combination formulation can be deduced to be due to the presence of chitosan in the formulation.

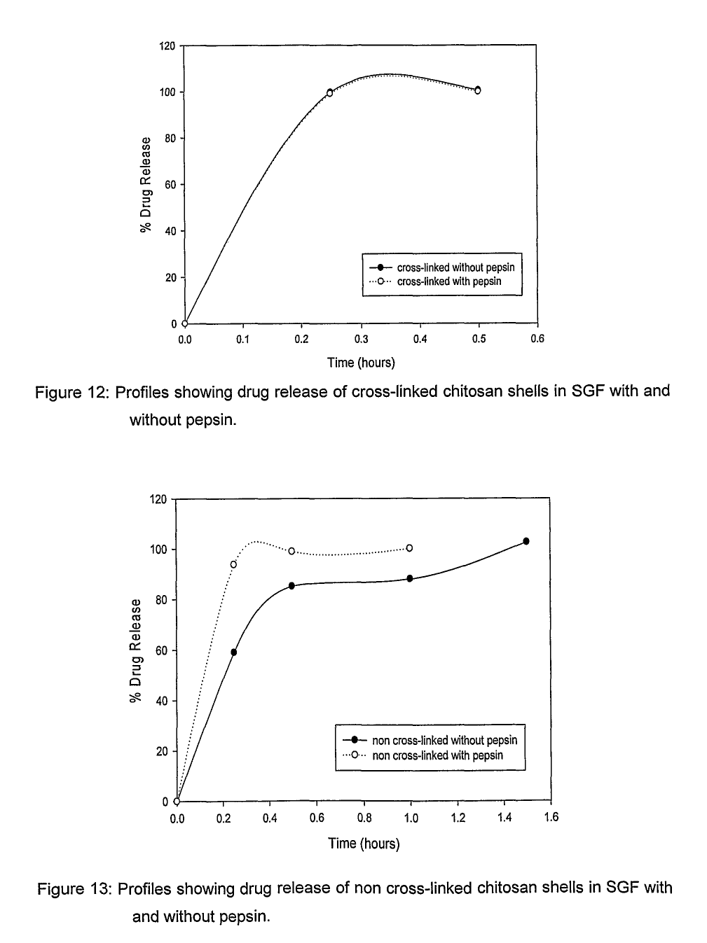

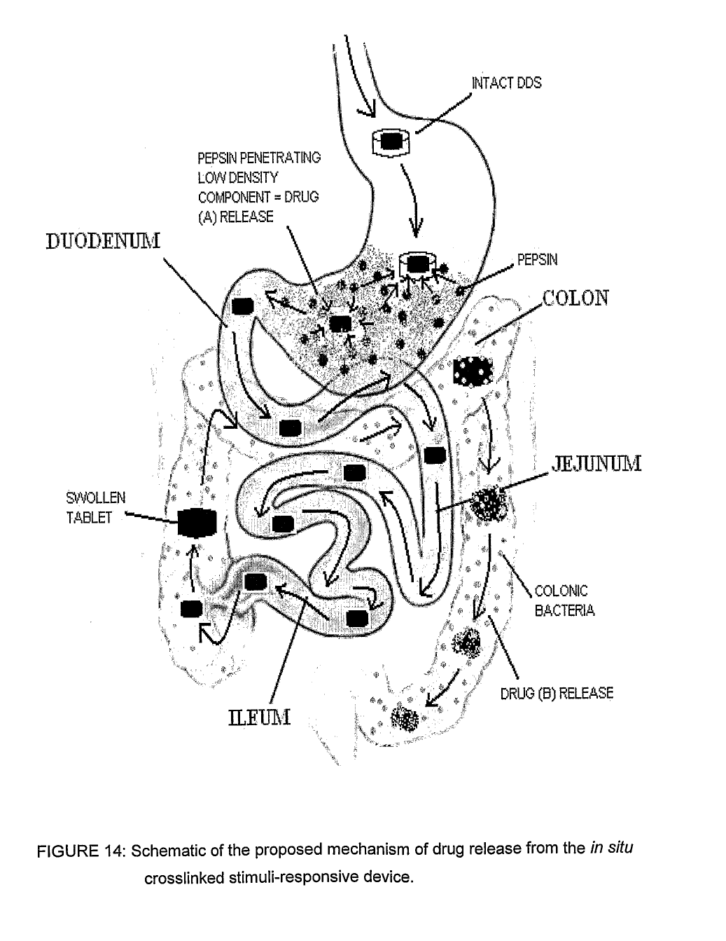

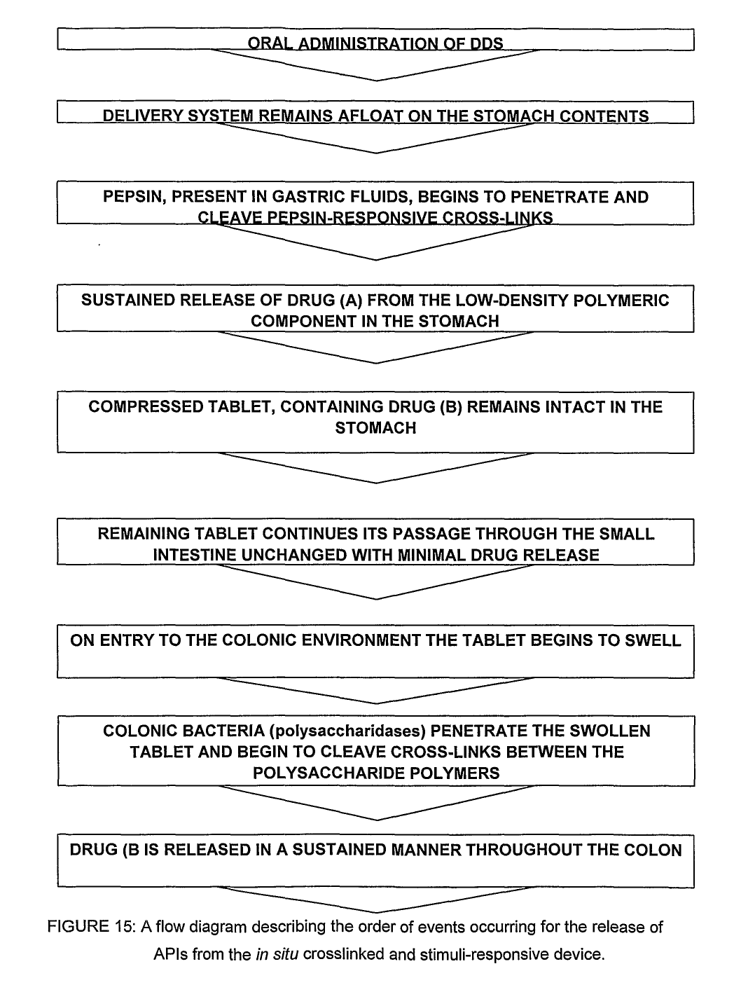

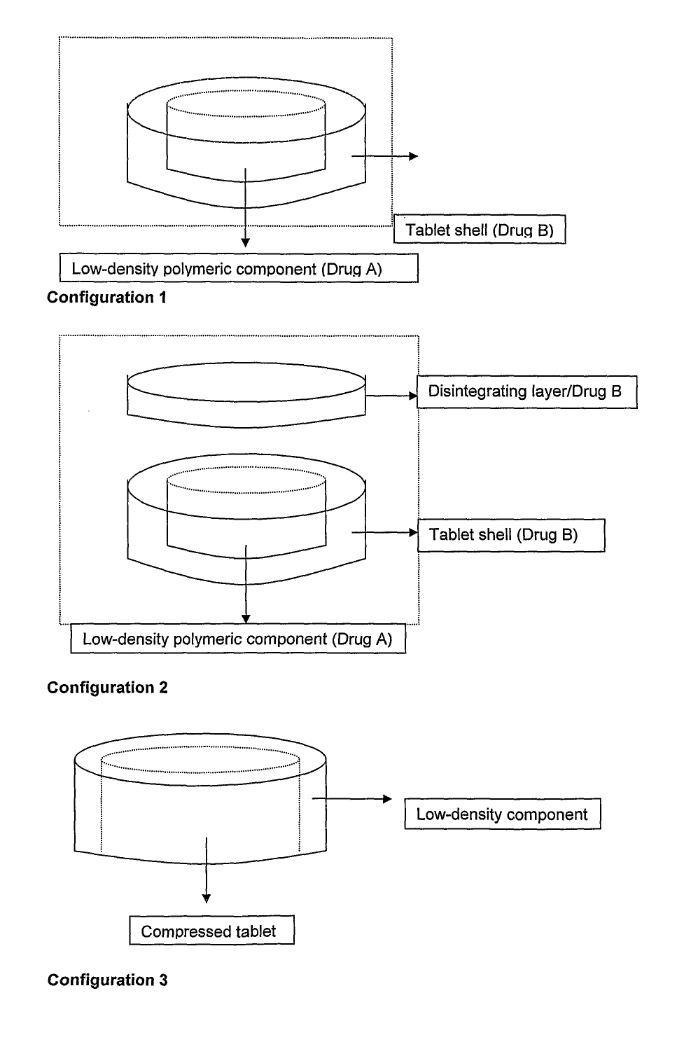

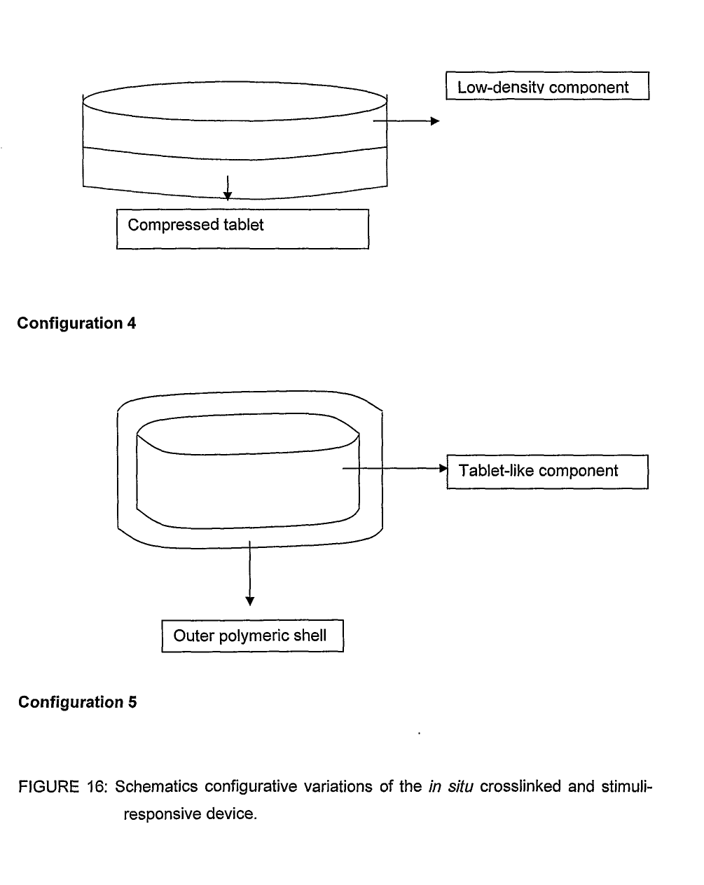

Crosslinked chitosan shells had the same drug release profiles in SGF with and without pepsin (FIG. 12).

FIG. 13 shows that altering the micro-environment of the chitosan shells results in a faster drug release in SGF with pepsin compared to SGF without pepsin throughout the release study, whereas the gelatin/chitosan formulations had only a brief period where drug release was increased in the presence of pepsin. This indicated the responsiveness of the non cross-linked chitosan shells to pepsin in SGF.

Naturally occurring polysaccharides are in abundance, are widely available, inexpensive and occur in varied structures with varying properties. Most polysaccharides are easily modifiable, and are highly stable, safe, non-toxic, hydrophilic and biodegradable. They are therefore ‘generally regarded as safe’ (GRAS) materials (Sinha and Kumria, 2003). Using polysaccharides as a means of delaying drug release in the gastro-intestinal tract is well-known however no products are yet available using this approach (Friend, 2005). The colon is an area of the gastro-intestinal tract where protein drugs are free from the attack of numerous proteases, and is thought to be an ideal location for the delivery of drugs into the bloodstream and the immune system. However they need to remain intact when travelling through the upper GI tract in order to protect the incorporated drugs from chemical and enzymatic degradation and they should be able to release the incorporated drugs immediately upon reaching the colon segment of the lower GI tract (Liu et al., 2003). Pectin is non-starch linear polysaccharide that remains intact in physiological conditions of the stomach and small intestine and is degraded by the bacterial inhabitants of the human large intestine and is therefore an ideal polymer for colon-targeted drug delivery. To reduce the aqueous solubility of pectin it has been used in the form of calcium pectinate (Sinha and Kumria, 2003). Cross-linking of pectin to salts retards the escape of drug from the cross-linked matrix and thus may prevent premature drug release.

This work has resulted in the successful design of an in situ crosslinked and stimuli-responsive device for site-specific delivery of multiple drugs in a single dosage form. In vitro studies have shown the potential for desirable release of drugs. These studies have also exhausted the possibilities of combinations between the polymers used, which led to further studies where different polymers/salts/other formulation excipients was introduced into the outer and inner platforms to control drug release.

1. A pharmaceutical dosage form for the site-specific delivery of more than one API, the dosage form comprising at least one outer layer containing at least one API for delivery to a first site in a human or animal body and at least one inner layer containing at least one API for delivery to a second site in the human or animal body, each layer having characteristics which, when subjected to specific stimuli unique to its delivery site, enable the release in said site of said API.

2. A pharmaceutical dosage form as claimed in claim 1 in which the dosage form has at least one intermediate layer located between the outer layer and the inner layer.

3. A pharmaceutical dosage form as claimed in claim 2 in which the intermediate layer contains at least one API for delivery to a site between the first and second sites.

4. A pharmaceutical dosage form as claimed in claim 1 in which the outer layer is in the form of a shell which, in use, inhibits release of APIs contained in the inner layers until substantially all of the API in the outer layer has been released.

5. A pharmaceutical dosage form as claimed in claim 4 in which each of the layers forms a platform and each API is incorporated into said platform.

6. A pharmaceutical dosage form as claimed in claim 5 in which the platforms are polymeric platforms.

7. A pharmaceutical dosage form as claimed in claim 6 in which the polymeric platforms are manufactured from natural and/or synthetic polymers.

8. A pharmaceutical dosage form as claimed in claim 7 in which the polymeric platforms are manufactured from natural polymers selected from the group of polysaccharide polymers.

9. A pharmaceutical dosage form as claimed in claim 8 in which the polysaccharide polymers are selected from the group consisting of: chitosan, pectin, xanthan gum, sodium alginate, celluloses, and dextrans.

10. A pharmaceutical dosage form as claimed in claim 7 in which the polymeric platforms are manufactured from synthetic polymers which include a standard hydrophilic polymer.

11. A pharmaceutical dosage form as claimed in claim 7 in which the polymeric platforms are manufactured from synthetic polymers which include a hydrophilic, swellable or erodible polymer.

12. A pharmaceutical dosage form as claimed in claim 7 in which the polymeric platforms are manufactured from synthetic polymers which include a standard hydrophobic polymer.

13. A pharmaceutical dosage form as claimed in claim 7 in which the polymeric platforms are manufactured from synthetic polymers which include a hydrophobic swellable or erodible polymer.

14. A pharmaceutical dosage form as claimed in claim 7 in which the polymeric platforms are manufactured from synthetic polymers which include a stimulus-responsive polymer.

15. A pharmaceutical dosage form as claimed in claim 7 in which the polymeric platforms are manufactured from polymers which include at least one of polyethylene oxide (PEO), polyvinyl alcohol (PVA), ethylcellulose (EC), poly(lactic) co-glycolic acids (PLGA), polylactic acids (PLA), polymethacrylates, polycaprolactones, polyesters and polyamides.

16. A pharmaceutical dosage form as claimed in claim 7 in which the polymeric platforms are manufactured from polymers which are mixed, in use, with a co-polymer.

17. A pharmaceutical dosage form as claimed in claim 7 in which the polymeric platforms are manufactured from polymers which are used on their own.

18. A pharmaceutical dosage form as claimed in claim 1 in which the or each API is in the form of micro- and/or nanostructures and these structures are incorporated into a polymeric platform by mixing them with the polymer and/or other rate-modulating critical formulation adjuvants.

19. A pharmaceutical dosage form as claimed in claim 1 in which the APIs are delivered to regions of the gastrointestinal tract.

20. A pharmaceutical dosage form as claimed in claim 19 in which the regions of the gastrointestinal tract to which the APIs are delivered are the stomach or the colon.

21. A pharmaceutical dosage form as claimed in claim 19 in which the pharmaceutical dosage form has gastrofloatable properties where it is initially buoyant or becomes buoyant on the surface of gastric contents thus preventing premature gastric emptying.

22. A pharmaceutical dosage form as claimed in claim 19 in which the pharmaceutical dosage form has gastrosinking properties where it is more dense than the gastric fluid and sinks to the antrum of the stomach in use.

23. A pharmaceutical dosage form as claimed in claim 19 in which the pharmaceutical dosage form has gastroswellable properties where the dosage form swells, in use, and prevents the rapid gastric emptying through the pyloric sphincter of the stomach based on swellable dimensions of the dosage form.

24. A pharmaceutical dosage form as claimed in claim 19 in which the pharmaceutical dosage form adheres, in use, to the wall of the stomach or another region of the GIT thus preventing premature gastric emptying, duodenal emptying, intestinal emptying, or colonic emptying depending on the site of adhesion.

25. A pharmaceutical dosage form as claimed in claim 5 in which the outer platform of the dosage form dissolves in response to site-specific stimuli, preferably pepsin, in the stomach and, once dissolved, the remainder of the dosage form moves, in use, into and past through the small intestine to, eventually, enter the colonic region of the gastrointestinal tract where the inner platform of the dosage form dissolves in response to site-specific stimuli in the colonic region and release the API.

26. A pharmaceutical dosage form as claimed in claim 25 in which the outer platform of the dosage form dissolves in response to pepsin in the stomach.

27. A pharmaceutical dosage form as claimed in claim 5 in which the inner platform of the dosage form contains and releases at least one compound that enhances absorption of the API in the colonic region.

28. A pharmaceutical dosage form as claimed in claim 5 in which the dosage form releases the API incorporated into each platform as the platform dissolves thus making the API available for absorption in the site in which it is released and/or making the API available to act locally at its target site.

29. A pharmaceutical dosage form as claimed in claim 28 in which the API is selected from one or more of several APIs which are selected from the group consisting of: anti-inflammatories, corticosteroids, antidiarrhoeals, opioids, immunosuppressives, antibiotics, antiemetics, antifungals, antivirals, antimalarials, anti-TB, antiretrovirals, antihypertensives, proteins, peptides, chemotherapeutics, diagnostic agents, probiotics, prebiotics, multivitamins, minerals, trace elements, and phytonutrients.

30. A pharmaceutical dosage form as claimed in claim 6 in which the polymers forming the polymeric platforms are in situ crosslinked with an electrolyte or salt which is incorporated into the pharmaceutical dosage form, the electrolyte or salt being selected from the Hofmeister Series of salts and operable to retard the release of APIs from the pharmaceutical dosage from any or all of the platforms and or glutaraldehyde and formaldehyde.

31. A pharmaceutical dosage form as claimed in claim 6 in which the polymer is crosslinked by using microwave radiation, UV radiation or chemical crosslinking.

32. A pharmaceutical dosage form as claimed in claim 1 in which the operatively innermost layer of the pharmaceutical dosage form is at least one in situ crosslinked polymer forming a single discrete pellet containing at least one API embedded therein.

33. A pharmaceutical dosage form as claimed in claim 1 in which the operatively innermost layer of the pharmaceutical dosage form has a number of in situ crosslinked polymers and for the polymer or polymers to form a polymer matrix of various stimuli-responsive polymers and/or other critical formulation adjuvants and desired permutations depending on the nature of the polymer or polymers selected.

34. A pharmaceutical dosage form as claimed in claim 1 in which the dosage form is formed by mixing a polymer in various concentrations, a pharmaceutical excipient and/or a binder and/or a crosslinking agent, and at least one active ingredient in at least one of the components of the dosage form.

35. A pharmaceutical dosage form as claimed in claim 34 in which the pharmaceutical excipient is a lubricant.

36. A pharmaceutical dosage form as claimed in claim 35 in which the release of each API from the outer polymeric layer of the pharmaceutical dosage form is governed by the crosslinking agent employed, the degree of ionization of the crosslinking agent, the solution pH, the ratio of dry polymer to pepsin, and the degree of crosslinking.

37. A pharmaceutical dosage form as claimed in claim 6 in which the innermost polymeric platform is configurable to suit a number of applications and administration methods.

38. A pharmaceutical dosage form as claimed in claim 6 in which the innermost polymeric platform is embedded within the outermost, gastrofloatable, polymeric platform so that, in use, APIs from either polymeric platform can be released over a desired period of time, preferably in a phase-controlled site-specific manner which may be rapid, alternatively slow, as a result of variations in the diffusion path lengths created within the polymeric platforms.

39. A pharmaceutical dosage form as claimed in claim 38 in which the outermost polymeric platform has a low density.

40. A pharmaceutical dosage form as claimed in claim 1 in which a pharmaceutically active compound is formulated into at least one disc and for the disc to be surrounded by a number of the same or alternating polymeric layers.

41. A pharmaceutical dosage form as claimed in claim 6 in which the outer polymeric platform is in the form of a shell which, wholly or partly encapsulates an inner tablet-like component, the outer polymeric platform thus allowing the release, in use, of a first API in one region of the gastrointestinal tract, in response to specific stimuli in said region of the gastrointestinal tract.

42. A pharmaceutical dosage form as claimed in claim 41 in which the composition of the shell comprises various natural and synthetic polymers.

43. A pharmaceutical dosage form as claimed in claim 42 in which the polymers are selected from the group consisting of chitosan, gelatin and polyacrylamide and the crosslinking agents comprise sucrose-6-1′-diacrylate.

44. A pharmaceutical dosage form as claimed in claim 43 in which the outer shell adheres to the inner tablet-like component using polymers with adhesive properties.

45. A pharmaceutical dosage form as claimed in claim 44 in which the tablet-like component comprises crosslinked API-loaded granules dispersed within a matrix of various natural and synthetic polymers.

46. A pharmaceutical dosage form as claimed in claim 45 in which the polymers are selected from pectin, polyethylene oxide (PEO), and xanthan gum.

47. A pharmaceutical dosage form as claimed in claim 46 in which the granules comprise natural polysaccharide polymers that are responsive, in use, to specific enzymes in various regions of the gastrointestinal tract.

48. A pharmaceutical dosage form as claimed in claim 47 in which the natural polysaccharide polymers are selected from the group consisting of alginate, pectin, xanthan gum and chitosan.

49. A pharmaceutical dosage form as claimed in claim 48 in which the polysaccharide polymers are susceptible to digestion/cleavage by colonic enzymes.

50. A pharmaceutical dosage form as claimed in claim 49 in which granule polymers are crosslinked with various electrolytes/salts or multivalent salts.

51. A pharmaceutical dosage form as claimed in any one of claim 49 in which the tablet-like matrix is in situ crosslinked using various crosslinking agents.

52. A pharmaceutical dosage form as claimed in claim 50 in which the tablet-like component is coated with a pH responsive, coating solution.

53. A pharmaceutical dosage form as claimed in claim 50 in which the tablet-like component is coated with a pH independent coating solution.

54. A pharmaceutical dosage form as claimed in claim 50 in which the tablet-like component is coated with at least one hydrophobic polymer latex selected from the group consisting of ethylcellulose, or cellulose acetate phthalate.

55. A pharmaceutical dosage form as claimed in claim 54 in which the coating solutions are aqueous dispersions.

56. A pharmaceutical dosage form as claimed in claim 54 in which the coating solutions are dispersed in solvents.

57. A pharmaceutical dosage form as claimed in claim 54 in which the hydrophobic polymers are dispersed within the matrix of the tablet-like component.

58. A pharmaceutical dosage form as claimed in claim 57 in which the pH responsive or pH-independent coating, solution or hydrophobic polymer latex is applied to the pharmaceutical dosage form alone or in combination.

59. A pharmaceutical dosage form as claimed in claim 58 in which the coating solutions are combined with various polysaccharide or enzyme responsive polymers in various ratios and combinations to form a desired pH/time/enzyme responsive coating.

60. A pharmaceutical dosage form as claimed in claim 59 in which the combination of coating solutions and polymers are selected so as to render a polymeric component of the pharmaceutical dosage form pH responsive in use, thus facilitating precise delivery of an API to a desired site of action or absorption.

61. A pharmaceutical dosage form as claimed in claim 59 in which the coating solutions and polymers are selected so as to render a polymeric component of the pharmaceutical dosage form responsive to one or more enzymes present in a desired site of action or absorption thus facilitating precise delivery of an API to a desired site of action or absorption.

62. A pharmaceutical dosage form as claimed in claim 59 in which the combination of coating solutions and polymers are selected to degrade within a specific region of the human or animal body in a time dependent manner thus facilitating precise delivery of an API to a desired site of action or absorption.

63. A pharmaceutical dosage form as claimed in claim 62 in which the inner polymeric layer of the pharmaceutical dosage form is tablet-shaped.

64. A pharmaceutical dosage form as claimed in claim 63 in which the coating solutions for the tablet are formed by compressing granules prepared by wet or dry granulation of a polysaccharide polymer or a combination of polymers and the API.

65. A pharmaceutical dosage form as claimed in claim 63 in which the inner polymeric layer of the pharmaceutical dosage form is formed by compressing granules of a polysaccharide polymer or a combination of polymers with a single or combination of crosslinking agents, using various solvents, and the API.

66. A pharmaceutical dosage form as claimed in claim 65 in which the granules are prepared by wet granulation methods.

67. A pharmaceutical dosage form as claimed in claim 65 in which the granules are prepared by dry granulation methods.

68. A pharmaceutical dosage form as claimed in claim 66 in which the granules are coated with a pH responsive or pH-independent coating solution or various hydrophobic polymer latexes which may be applied alone or in combination or not in a matrix of a single polymer or a combination of polymers.

69. A pharmaceutical dosage form as claimed in claim 63 in which the tablet-shaped inner polymeric layer of the pharmaceutical dosage form is formed by direct compression of polymeric components of the formulation.

70. A pharmaceutical dosage form as claimed in claim 52 in which the coating or coatings of the dosage form are combined with various polysaccharide or enzyme responsive polymers in various ratios and combinations to form a unique pH/time/enzyme responsive coating.

71. A pharmaceutical dosage form as claimed in claim 6 in which the micro-environment of the outer polymeric shell is altered to facilitate an optimum environment for the chitosanolytic activity of pepsin, in use, thus improving the enzymatic responsiveness of the outer polymeric shell and ensuring sufficient or complete and site-specific delivery of the API.

72. A pharmaceutical dosage form as claimed in claim 6 in which the micro-environment of the outer polymeric shell is altered by adding various alkaline solutions or by employing salts directly.

73. (canceled)

74. A pharmaceutical dosage form as claimed in claim 9 in which the celluloses is sodium carboxymethycellulose (CMC), hydroxypropylcellulose (HPC), hydroxyethylcellulose (HEC) or hydroxypropylmethylcellulose (HPMC).

75. A pharmaceutical dosage form as claimed in claim 34 in which the binder is carboxymethylcellulose (CMC) and the crosslinking agent is a desired salt.

76. A pharmaceutical dosage form as claimed in claim 35 in which the lubricant is magnesium stearate.

77. A pharmaceutical dosage form as claimed in claim 41 in which the specific stimuli in said region of the gastrointestinal tract is pepsin.

78. A pharmaceutical dosage form as claimed in claim 41 in which the region of the gastrointestinal tract where the first API is released is the stomach.

79. A pharmaceutical dosage form as claimed in claim 44 in which the polymers with adhesive properties comprise polyvinylalcohol (PVA).

80. A pharmaceutical dosage form as claimed in claim 47 in which the particular region of the gastrointestinal tract is a colon.

81. A pharmaceutical dosage form as claimed in claim 49 in which the colonic enzymes are β-glucosidases, pectinases or other polysaccharidases.

82. A pharmaceutical dosage form as claimed in claim 50 in which the electrolytes/salts are multivalent salts.

83. A pharmaceutical dosage form as claimed in claim 82 in which the multivalent salts are tripolyphosphates.

84. A pharmaceutical dosage form as claimed in any one of claim 51 in which the crosslinking agents are electrolytes/salts.

85. A pharmaceutical dosage form as claimed in claim 56 in which the solvents are acetone or ethanol.

86. A pharmaceutical dosage form as claimed in claim 65 in which the crosslinking agents are multivalent salts or other chemical reagents.

87. A pharmaceutical dosage form as claimed in claim 65 in which the solvents are ionized water or ethanol.

88. A pharmaceutical dosage form as claimed in claim 67 in which the granules are coated with a pH responsive or pH-independent coating solution or various hydrophobic polymer latexes which may be applied alone or in combination or not in a matrix of a single polymer or a combination of polymers.

89. A pharmaceutical dosage form as claimed in claim 72 in which the alkaline solutions are selected from sodium hydroxide solutions of various concentrations and ammonium hydroxide solutions of various concentrations.

90. A pharmaceutical dosage form as claimed in claim 72 in which the salts are sodium bicarbonate, sodium carbonate or a combination thereof.

91. A pharmaceutical dosage form as claimed in claim 75 in which the charge densities of the relevant polymers and crosslinking salts of the said pharmaceutical dosage form are governed by the solution pH, with a lower solution pH producing, in use, a sufficient decrease in the degree of ionization of the crosslinking salt which results in polymeric crosslinking weakening between the polymer/s and crosslinking agent/s to facilitate the swelling of the outer polymeric layer of the pharmaceutical dosage form and allow for diffusion of fluid along with pepsin into the layer and cause cleavage and/or degradation of chitosan which results in API release from the pharmaceutical dosage form.
